(19) United States
(12) Patent Application Publication
(10) Pub. No.: US 2023/0270452 A1
(43) Pub. Date: Aug. 31, 2023

(54) Method of Fusing a Tibiotalar Joint and Fused Tibiotalar Joint

(71) Applicant: University of Kentucky Research Foundation, Lexington, KY (US)

(72) Inventors: Arun Aneja, Lexington, KY (US); Lorenzo Deveza, Houston, TX (US); Arjun Srinath, Lexington, KY (US); Eric Abbenhaus, Lexington, KY (US)

(21) Appl. No.: 18/003,802

(22) PCT Filed: Jun. 28, 2021
(86) PCT No.: PCT/US2021/039379

§ 371 Date: Dec. 29, 2022

Related U.S. Application Data
(60) Provisional application No. 63/045,374, filed on Jun. 29, 2020.

(51) Int. Cl. A61B 17/17; A61B 17/72; A61F 2/30

(52) U.S. Cl. CPC A61B 17/1725; A61B 17/72; A61F 2/30; A61F 2/4202

(57) ABSTRACT

A method for fusing a tibiotalar joint of a patient with an intramedullary device while leaving an adjacent subtalar joint intact includes the steps of placing an intramedullary nail through a talus and into a tibia of the patient without violating a posterior facet of the adjacent subtalar joint and fixing the intramedullary nail to the talus and the tibia.

### RELATED APPLICATION

This application claims priority to U.S. Provisional Patent Application Ser. No. 63/045,374 filed on Jun. 29, 2020, which is hereby incorporated by reference in its entirety.

### TECHNICAL FIELD

This document relates to the field of fracture stabilization, ankle arthritis, and joint arthrodesis. More particularly, this document pertains to the fusion of the tibiotalar joint with an intramedullary device while leaving the talocalcaneal or subtalar joint intact.

### BACKGROUND

The incidence of geriatric ankle fractures is increasing with the aging population, there is no consensus on the optimal management of these fractures. Current treatment methods of open reduction are invasive and require prolonged periods of immobilization. Tibio-talar-calcaneal nails violate the subtalar joint. A tibio talar nail would allow for fracture stabilization and immediate weight bearing.

When operative intervention is used to treat ankle arthritis, there are two main options: ankle replacement and ankle fusion. Ankle fusion can be performed using either a plate or intramedullary nail. The benefit of using a plate for ankle fusion is that you can isolate the fusion to the tibiotalar (TT) joint and leave the talo-calcaneal (TC) joint untouched. However, the use of a plate requires more extensive dissection and the implant does not provide enough stability to allow for immediate weight bearing. The benefit of using a nail for tibiotalar fusion is that it allows for immediate weight bearing and requires less dissection for exposure. The downside to modern intramedullary nails currently used for ankle fusion is that the implant traverses the calcaneus, talus, and tibia, resulting in a fusion between the subtalar joint which is often unnecessary.

Patients who undergo tibiotalar joint fusion with a plate are generally non-weight bearing for 6 weeks or longer after surgery. This period of non-weight bearing results in muscle atrophy, deconditioning, and increases risk of deep vein thrombosis (DVT) or pulmonary embolism (PE). These patients are also typically casted for 8-12 weeks after surgery, which can lead to skin irritation or breakdown. The increased dissection required for plate placement leads to increased disruption of blood supply and wounds which take longer to heal, which is particularly troublesome in diabetic patients who represent a growing segment of patients needing ankle fusion.

While patients who undergo tibiotalar and subtalar fusion get the benefit of immediate weight bearing and easier healing wounds, they must deal with the aftermath of a fused subtalar joint. Studies have demonstrated that subtalar fusion results in loss of motion and gait instability. A normal subtalar joint is capable of subtalar compensation, resulting in improved lower extremity alignment in the setting of ankle or knee deformity. Fusion of the subtalar joint results in a loss of subtalar compensation, exacerbating or even unmasking issues caused by deformity in the knee or ankle.

The ideal implant for ankle fusion would be an intramedullary nail that did not also fuse the subtalar joint. The tibiotalar fusion nail allows for immediate weight bearing, causes minimal soft tissue disruption, and preserves subtalar compensation by leaving the subtalar joint intact.

A cadaveric study was performed demonstrating safety of inserting the tibiotalar fusion nail, as flexor hallucis longus (FHL) was not disrupted. Furthermore, the study showed that the subtalar joint remained intact. Based on the nail entry site, implanting the tibiotalar fusion nail is also safe for the lateral plantar nerve and artery.

### SUMMARY

In accordance with the purposes and benefits described herein, a method is provided for fusing a tibiotalar joint of a patient with an intramedullary device while leaving adjacent talocalcaneal or subtalar joint intact. That method comprises the steps of: (a) placing an intramedullary nail through a talus and into a tibia of the patient without violating a posterior facet of the adjacent subtalar joint and (b) fixing the intramedullary nail to the talus and the tibia.

In one or more embodiments, the method further includes the step of compressing the tibiotalar joint. In one or more embodiments, the method further includes the step of using a guidewire for the placing of the intramedullary nail. The method may also include starting the guidewire just medial to a main body of a calcaneus at a planar aspect of a sustentaculum tali. Further, the method may include the step of inserting the guidewire through the calcaneus into the talus and the tibia.

The method may include the step of reaming the talus and the tibia over the guidewire by advancing through the sustentaculum tali and into an inferior aspect of the talus without violating the posterior facet of the subtalar joint. The method may also include the step of inserting the intramedullary nail into the talus and the tibia through the sustentaculum tali.

In one or more embodiments, the method includes the step of making a plantar incision over the guidewire and placing a retractor medial to a sustentaculum to protect the flexor hallucis longus tendon and the adjacent neurovascular bundle prior to the reaming. The method may also include the step of placing a drill sleeve over the guidewire prior to reaming in order to protect soft tissue.

Still further, the method may include one or more of any of the following steps:
a. using an outrigger to place a plurality of screws for the fixing of the intramedullary nail to the talus and the tibia;
b. placing a compression screw in the tibia and the talus with the outrigger outside of the intramedullary nail;
c. placing multiple interlocking screws proximally in the nail and the tibia;
d. placing a screw through the intramedullary nail into the talus in a trans malleolar fashion or any other appropriate plane for stability; and
e. inserting the intramedullary nail into the talus and the tibia through the calcaneus.

In accordance with yet another aspect, a method for fusing a tibiotalar joint of a patient with an intramedullary device while leaving the adjacent talocalcaneal or subtalar joint intact, comprises: (a) inserting an intramedullary nail through the calcaneus just medial to a main body of a calcaneus at a planar aspect of a sustentaculum tali into a talus and a tibia of the patient without violating a posterior facet of the adjacent subtalar joint and (b) fixing the intramedullary nail to the talus and the tibia.

In one or more embodiments, the method further includes the step of compressing the tibiotalar joint. In one or more embodiments, the method further includes the step of using a guidewire for the placing of the intramedullary nail. The method may also include starting the guidewire just medial to a main body of a calcaneus at a planar aspect of a sustentaculum tali. Further, the method may include the step of inserting the guidewire into the talus and the tibia.

The method may include the step of reaming the talus and the tibia over the guidewire by advancing through the sustentaculum tali and into an inferior aspect of the talus without violating the posterior facet of the subtalar joint. The method may include the step of making a plantar incision over the guidewire and placing a retractor medial to a sustentaculum to protect the flexor hallucis longus tendon and the adjacent neurovascular bundle prior to the reaming. The method may also include the step of placing a drill sleeve over the guidewire prior to reaming in order to protect soft tissue.

Still further, the method may include one or more of any of the following steps:
a. using an outrigger to place a plurality of screws for the fixing of the intramedullary nail to the talus and the tibia;
b. placing a compression screw in the tibia and the talus with the outrigger outside of the intramedullary nail;
c. placing multiple interlocking screws proximally in the nail and the tibia;
d. placing a screw through the intramedullary nail into the talus in a trans malleolar fashion or any other appropriate plane for stability; and
e. inserting the intramedullary nail into the talus and the tibia through the calcaneus

In accordance with yet another aspect, a fused tibiotalar joint, comprises an intramedullary nail extending through a talus and into a tibia of the patient without violating a posterior facet of the adjacent talocalcaneal or subtalar joint.

The fused tibiotalar joint may include at least one screw fixing the intramedullary nail to the tibia. That screw may be placed proximally.

The fused tibiotalar joint may include at least one screw fixing the intramedullary nail to the talus. That screw may be placed in a trans malleolar fashion for stability.

The fused tibiotalar joint may include a compression screw fixed in the tibia and the talus outside of the intramedullary nail. Alternatively, the fused tibiotalar joint may include a compression screw built within the intramedullary nail at the proximal extent of the intramedullary nail.

In the following description, there are shown and described several preferred embodiments of the method of fusing a tibiotalar joint with an intramedullary nail while leaving the talocalcaneal or subtalar joint intact and the resulting fused tibiotalar joint. As it should be realized, the method and fused tibiotalar joint are capable of other, different embodiments and their several details are capable of modification in various, obvious aspects all without departing from the method and fused tibiotalar joint as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate several aspects of the method of fusing a tibiotalar joint with an intramedullary nail while leaving the talocalcaneal or subtalar joint intact and the resulting fused tibiotalar joint and together with the description serve to explain certain principles thereof.

FIG. 1 is an illustration of the anatomy of the ankle including the tibia, the talus, the calcaneus, the tibiotalar joint and the subtalar joint.

FIG. 2 is a posterior view of the anatomy of the ankle clearly showing the sustenaculum tali of the calcaneus and the insertion point of the intramedullary nail.

FIG. 3 is a side elevational view of the intramedullary nail used for tibiotalar joint fusion.

FIG. 4 is an elevational view illustrating how the outrigger is connected to the nail of FIG. 3 to allow for alignment and placement of the interlocking screws in the fastener receivers of the nail.

FIG. 5 is a detailed view of one possible alternative embodiment of the intramedullary nail including an internal screw that is used to provide compression to the tibiotalar joint following placement of the nail across that joint.

FIGS. 6A and 6B are respective rear and side elevational views illustrating the intramedullary nail implanted in the talus and tibia and connected to the outrigger that guides the placement of the interlocking screws that hold the nail in position fusing the tibiotalar joint and providing desired compression to that joint.

FIGS. 7A and 7B correspond to FIGS. 6A and 6B with the exception that the outrigger has been disconnected from the nail and removed and the nail is locked in position fusing the tibiotalar joint while leaving the subtalar joint intact.

Reference will now be made in detail to the present preferred embodiments of the method of fusing a tibiotalar joint with an intramedullary nail while leaving the talocalcaneal joint or subtalar intact as well as the resulting fused tibiotalar joint, examples of which are illustrated in the accompanying drawing figures.

### DETAILED DESCRIPTION

Accordingly, it is to be understood that the embodiments of the method of fusing a tibiotalar joint with an intramedullary nail while leaving the talocalcaneal or subtalar joint intact and the resulting fused tibiotalar joint 10 set forth and described herein are merely illustrative and not restrictive. Reference herein to details of the illustrated embodiments is not intended to limit the scope of the claims. As used herein, the term “and/or” includes “and” and all combinations of one or more of the associated listed items. As used herein, the singular forms “a”, “an,” and “there” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this device and method belong. It will be further understood that terms, such as those defined in commonly used dictionaries, should be interpreted as having a meaning that is consistent with their meaning in the context of the relevant art and the present disclosure and will not be interpreted in an idealized overly formal sense unless expressly so defined herein.

In describing the method and the fused tibiotalar joint 10, it will be understood that a number of techniques and steps are disclosed. Each of these have individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of this document and the claims.

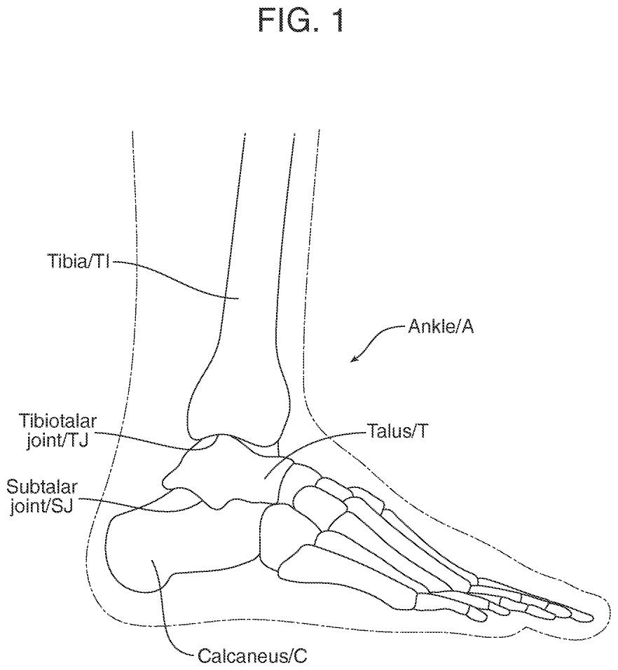

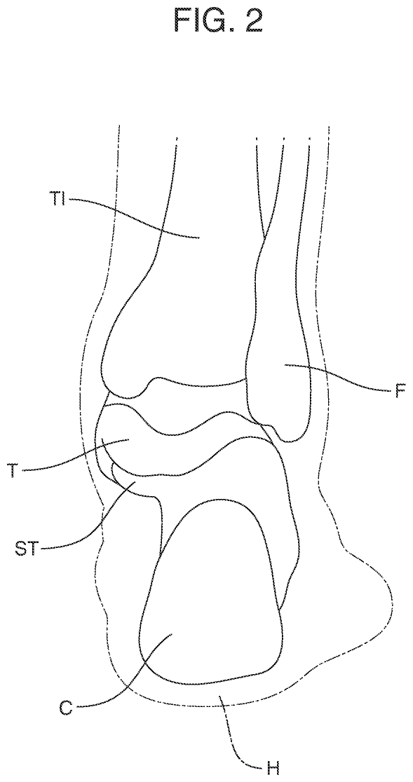

Reference is now made to FIGS. 1 and 2 which illustrate the anatomy of the human ankle A and clearly shows the bones of the ankle including the tibia TI, the talus T and the calcaneus C. The tibia TI and the talus T form the tibiotalar joint TJ at their interface while the talus and the calcaneus C form the subtalar joint SJ at their interface.

FIG. 2 clearly shows the insertion point for the intramedullary nail 12 at the the sustenaculum tali ST of the calcaneus C. As will become apparent from the following description, when the nail 12 is properly implanted it engages and is locked to the talus T and the tibia TI in order to fuse the tibiotalar joint TJ while being free of the calcaneus C so as to leave the subtalar joint ST between the talus and the calcaneus intact and operative.

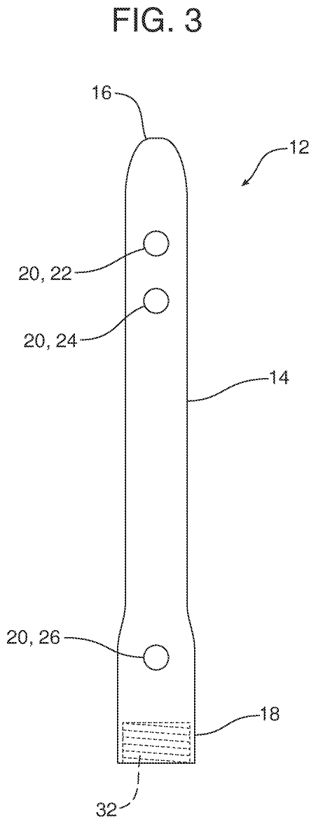

Reference is now made to FIGS. 3-5 which illustrate the tibiotalar nail implant system 10 which includes the intramedullary nail 12 comprising a hollow, elongated body 14 including a rounded or tapered first end 16 and a slightly enlarged second end 18. The first end 16 is adapted for insertion into a bore B that is drilled through the sustenaculum tali ST of the calcaneus C and the talus T into the tibia TI in a manner described in detail below. This is done while leaving the subtalar joint SJ between the talus T and the calcaneus C fully intact.

As best illustrated in FIGS. 3, the nail 12 includes a plurality of fastener receivers 20 including, particularly, (a) a first aperture 22 adjacent the first end 16 (b) a second aperture 24 adjacent the first aperture at the first end of the nail and a third aperture 26 adjacent the second end 18.

The nail 12 may be constructed from implantable stainless steel alloys but could also be constructed of implantable grade titanium alloys, as well. Other material having the requisite properties of strength and inertness may be used.

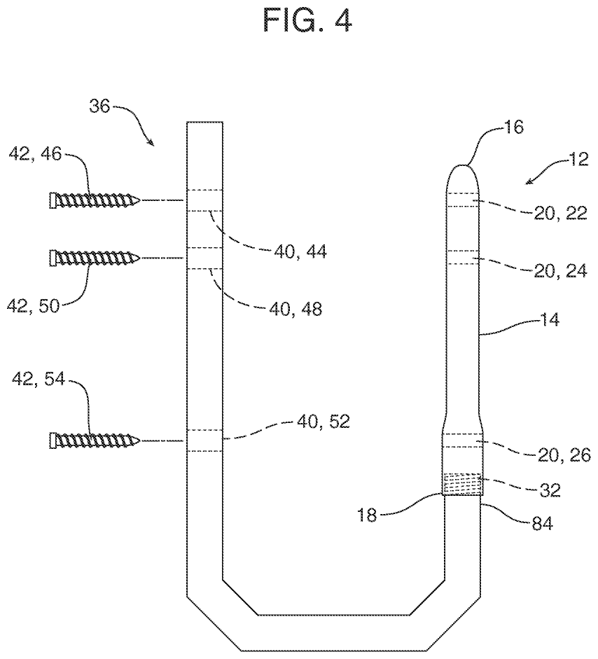

As illustrated in FIG. 4, the tibiotalar nail implant system 10 also includes an outrigger 36. In the illustrated embodiment, the outrigger 36 is substantially J-shaped. A distal end 38 of the outrigger 36 is adapted for connection in the second end 18 of the nail 12 by friction fit, threaded connection 32 shown in FIG. 3 or other means. The outrigger 36 includes a plurality of fastener locators 40 adapted for aligning a plurality of fasteners 42 with the plurality of fastener receivers 20 in the nail 12. The outrigger 36 may be made from materials similar to the nail 12 as described above. In the illustrated embodiment, the fasteners are interlocking screws.

In the illustrated embodiment, (a) the first fastener locator 44 aligns the first interlocking screw 46 with the first aperture 22, (b) the second fastener locator 48 aligns the second interlocking screw 50 the second aperture 24 and (c) the third fastener locator 52 aligns the third interlocking screw 54 with the third aperture 26. A guide sleeve (not shown), of a type known in the art, may be inserted into each fastener locator 40 to aid in the placement of the fasteners 42 in a manner known in the art (see US 2020/0113609).

In at least one possible embodiment, the tibiotalar nail implant system 10 includes a outrigger extension 56 that is configured with a compression screw guide 58 for placement of a compression screw 60 through the tibia TI into the talus T across the tibiotalar joint TJ outside of the nail 12. When tightened, the compression screw 60 provides compression to the then fused tibiotalar joint TJ in a manner described in greater detail below. The compression screw guide 58 may, for example, comprise a channel, an alignment aperture or a sleeve.

The tibiotalar implant system 10 described above is useful in a method of fusing a tibiotalar joint TJ with an intramedullary nail 12 while leaving the talocalcaneal of subtalar joint intact. Incisions should be thoughtfully planned, and the soft tissues should be handled with care. Joint preparation should be thorough and meticulous, and broad, congruent, bleeding cancellous surfaces should be created, ideally so that apposition of those surfaces can be obtained. All articular cartilage should be removed, as should the subchondral bone. Fixation of the arthrodesis site should be rigid. Particular attention should be paid to the position and alignment of the arthrodesis.

The following briefly describes the surgical approach and nail placement.
1. Under Flouroscopic guidance a guidewire is placed just medial to the main body of the calcaneus at the plantar aspect of the sustentaculum tali.
2. The wire is then inserted into the talus and the tibia.
3. A four cm plantar incision is made over the guidewire.
4. A retractor is placed medial to the sustentaculum to protect the FHL tendon and neurovascular bundle.
5. A drill sleeve to protect the soft tissue us placed over the guidewire.
6. A reamer is then used to ream over the guide wire.
7. The appropriate sized nail is placed.
8. At this point if compression is desired, the outrigger extension for the posterior to anterior compression screw is used. This guide allows placement of the compression screw outside of the nail.
9. Proximal interlocking screws are then placed proximally in the nail.
10. Then a screw is placed in the talus in a trans malleolar fashion for stability. If further compression is desired, then the talar screw can be placed in compression mode.

This disclosure may be considered to relate to the following items:
1. A method for fusing a tibiotalar joint of a patient with an intramedullary device while leaving adjacent subtalar joint intact, comprising:
placing an intramedullary nail through a talus and into a tibia of the patient without violating a posterior facet of the adjacent subtalar joint; and fixing the intramedullary nail to the talus and the tibia.
2. The method of item 1, including compressing the tibiotalar joint.
3. The method of item 2, including using a guidewire for the placing of the intramedullary nail.
4. The method of item 3, including starting the guidewire just medial to a main body of a calcaneus at a planar aspect of a sustentaculum tali.
5. The method of item 4, including inserting the guidewire through the calcaneus into the talus and the tibia.
6. The method of item 5, including reaming the talus and the tibia over the guidewire by advancing through the sustentaculum tali and into an inferior aspect of the talus without violating the posterior facet of the subtalar joint.
7. The method of item 6, including inserting the intramedullary nail into the talus and the tibia through the calcaneus so that the intramedullary nail is free and clear of the calcaneus.
8. The method of item 7, including making a plantar incision over the guidewire and placing a retractor medial to a sustentaculum to protect a flexor hallucis longus tendon and an adjacent neurovascular bundle prior to the reaming.
9. The method of item 8, including placing a drill sleeve over the guidewire prior to reaming in order to protect soft tissue.
10. The method of item 9, including using an outrigger to place a plurality of screws for the fixing of the intramedullary nail to the talus and the tibia.
11. The method of item 10, including placing a compression screw in the tibia and the talus with the outrigger outside of the intramedullary nail.
12. The method of item 11, including placing multiple interlocking screws proximally in the nail and the tibia.
13. The method of item 12, including placing a screw through the intramedullary nail into the talus in a trans malleolar fashion or any other appropriate plane for stability.
14. The method of any of items 1-5, including inserting the intramedullary nail into the talus and the tibia through the calcaneus.
15. The method of any of items 1-8, including using an outrigger to place a plurality of screws for the fixing of the intramedullary nail to the talus and the tibia.
16. The method of item 15, including placing a compression screw in the tibia and the talus outside of the intramedullary nail.
17. The method of item 16, including placing multiple interlocking screws proximally in the nail and the tibia.
18. The method of item 17, including placing a screw through the intramedullary nail into the talus in a trans malleolar fashion for stability.
19. The method of any of items 1-8, including placing a screw through the intramedullary nail into the talus in a trans malleolar fashion for stability.
20. A method for fusing a tibiotalar joint of a patient with an intramedullary device while leaving adjacent subtalar joint intact, comprising:
inserting an intramedullary nail through the calcaneus just medial to a main body of a calcaneus at a planar aspect of a sustentaculum tali into a talus and a tibia of the patient without violating a posterior facet of the adjacent talocalcaneal joint; and fixing the intramedullary nail to the talus and the tibia.
21. The method of item 20, including compressing the tibiotalar joint.
22. The method of item 21, including using a guidewire for placing of the intramedullary nail.
23. The method of item 22, including starting the guidewire just medial to a main body of a calcaneus at a planar aspect of a sustentaculum tali and inserting the guidewire through the calcaneus into the talus and the tibia.
24. The method of item 23, including reaming the talus and the tibia over the guidewire by advancing through the sustentaculum tali and into an inferior aspect of the talus without violating the posterior facet of the subtalar joint.
25. The method of item 24, including making a plantar incision over the guidewire and placing a retractor medial to a sustentaculum to protect a flexor hallucis longus tendon and an adjacent neurovascular bundle prior to the reaming.
26. The method of item 25, including placing a drill sleeve over the guidewire prior to reaming in order to protect soft tissue.
27. The method of item 26, including using an outrigger to place a plurality of screws for the fixing of the intramedullary nail to the talus and the tibia.
28. The method of item 27, including placing a compression screw in the tibia and the talus with the outrigger outside of the intramedullary nail.
29. The method of item 28, including placing multiple interlocking screws proximally in the nail and the tibia.
30. The method of item 29, including placing a screw through the intramedullary nail into the talus in a trans malleolar fashion for stability.
31. The method of any of items 20-28, including placing a screw through the intramedullary nail into the talus in a trans malleolar fashion for stability.
32. The method of any of items 20-25, including using an outrigger to place a plurality of screws for the fixing of the intramedullary nail to the talus and the tibia.
33. The method of item 32, including placing a compression screw in the tibia and the talus outside of the intramedullary nail.
34. The method of item 33, including placing two interlocking screws proximally in the nail and the tibia.
35. The method of item 34, including placing a screw through the intramedullary nail into the talus in a trans malleolar fashion for stability.
36. A fused tibiotalar joint, comprising:
an intramedullary nail extending through a talus and into a tibia of the patient without violating a posterior facet of the adjacent subtalar joint.
37. The fused tibiotalar joint of item 36, further including at least one screw fixing the intramedullary nail to the tibia.
38. The fused tibiotalar joint of item 37, further including at least one screw fixing the intramedullary nail to the talus.
39. The fused tibiotalar joint of item 38, wherein the at least one screw fixing the intramedullary nail to the talus is placed in a trans malleolar fashion for stability.
40. The fused tibiotalar joint of item 39 wherein the at least one screw fixing the intramedullary nail to the tibia is placed proximally.
41. The fused tibiotalar joint of any of items 36-40, further including a compression screw fixed in the tibia and the talus outside of the intramedullary nail.

Terms of approximation, such as the terms about, substantially, approximately, etc., as used herein, refers to ±10% of the stated numerical value. Use of the terms parallel or perpendicular are meant to mean approximately meeting this condition, unless otherwise specified.

It is to be fully understood that certain aspects, characteristics, and features, of the method of fusing a tibiotalar joint with an intramedullary nail while leaving the talocalcaneal joint intact and the resulting fused tibiotalar joint 10, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the method of fusing a tibiotalar joint with an intramedullary nail while leaving the talocalcaneal joint intact and the resulting fused tibiotalar joint 10 which are illustratively described and presented in combination or sub-combination in the context or format of a single embodiment may also be illustratively described and presented in the context or format of a plurality of separate embodiments.

Although method of fusing a tibiotalar joint with an intramedullary nail while leaving the talocalcaneal joint intact and the resulting fused tibiotalar joint 10 of this disclosure have been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

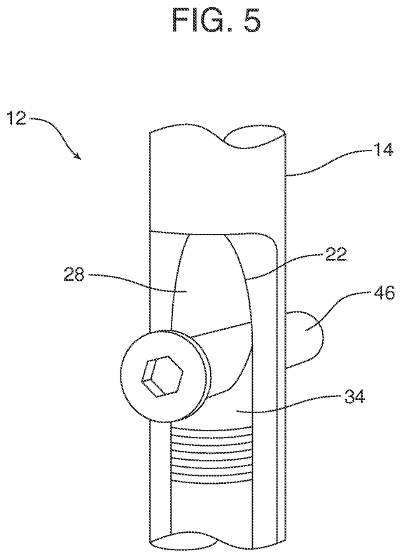

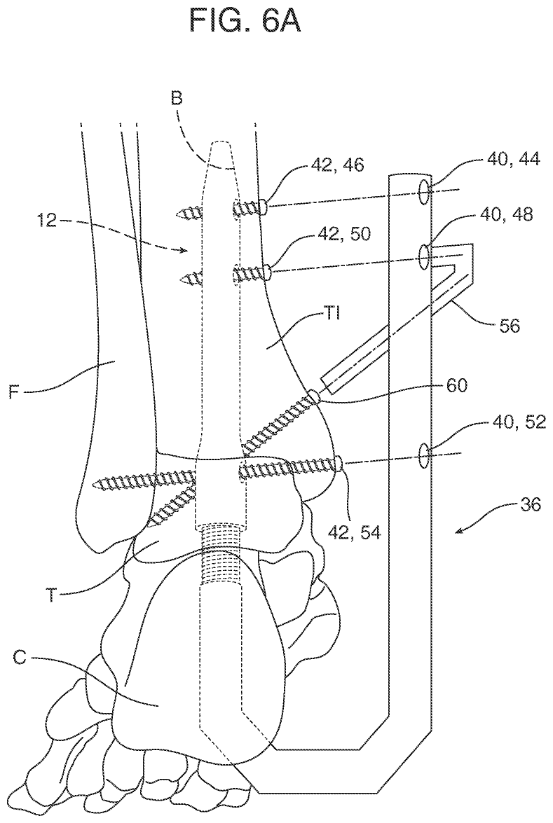

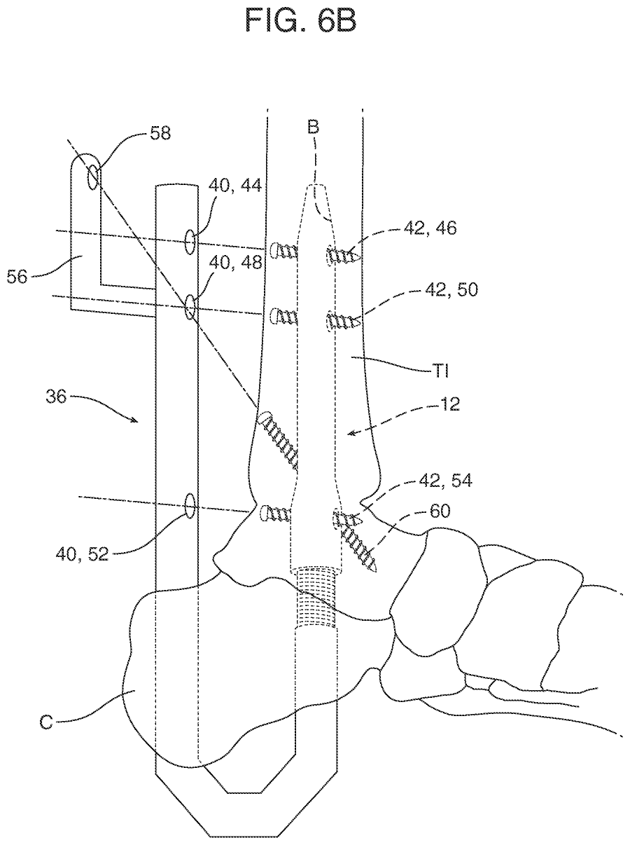

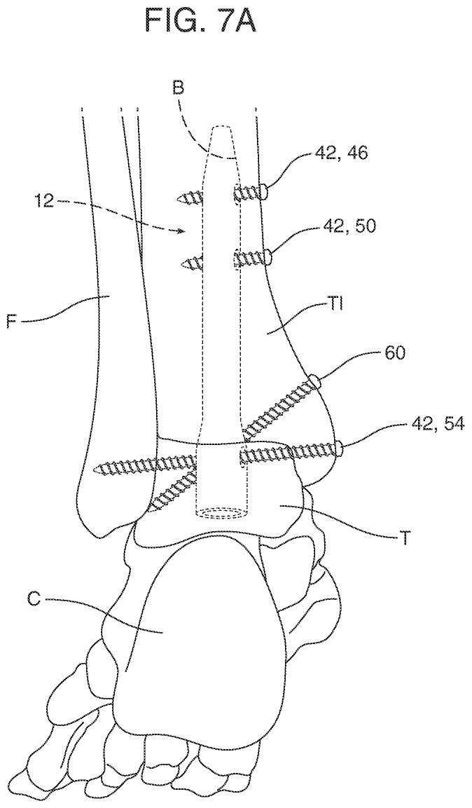

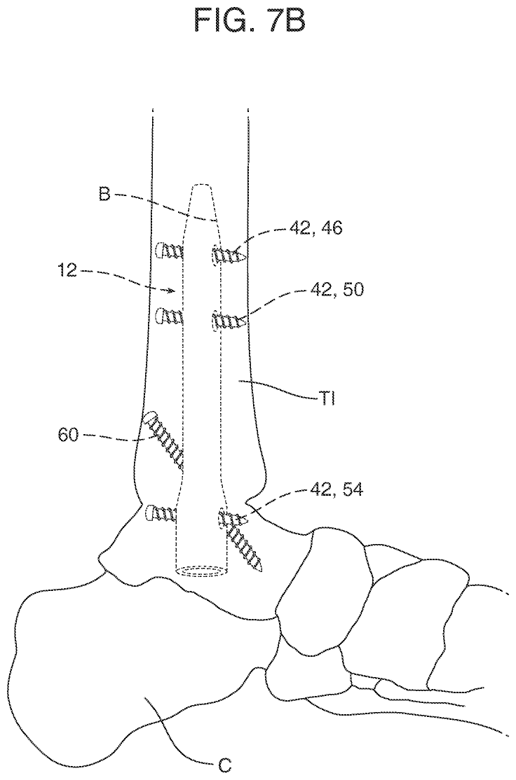

The foregoing has been presented for purposes of illustration and description. It is not intended to be exhaustive or to limit the embodiments to the precise form disclosed. Obvious modifications and variations are possible in light of the above teachings. For example, as illustrated in FIG. 5, the first aperture 22, at the first end 16 of the nail 12 could be an elongated slot and an internal screw 34 received in the lumen 28 of the nail at the second end 18 could be tightened against the first screw 46 in order to provide compression to the tibiotalar joint TJ instead of using the compression screw 60 as described above. All such modifications and variations are within the scope of the appended claims when interpreted in accordance with the breadth to which they are fairly, legally and equitably entitled.

1. A method for fusing a tibiotalar joint of a patient with an intramedullary device while leaving adjacent subtalar joint intact, comprising:
placing an intramedullary nail through a talus and into a tibia of the patient without violating a posterior facet of the adjacent subtalar joint; and fixing the intramedullary nail to the talus and the tibia.

2. The method of claim 1, including compressing the tibiotalar joint.

3. The method of claim 2, including using a guidewire for the placing of the intramedullary nail.

4. The method of claim 3, including starting the guidewire just medial to a main body of a calcaneus at a planar aspect of a sustentaculum tali.

5. The method of claim 4, including inserting the guidewire through the calcaneus into the talus and the tibia.

6. The method of claim 5, including reaming the talus and the tibia over the guidewire by advancing through the sustentaculum tali and into an inferior aspect of the talus without violating the posterior facet of the subtalar joint.

7. The method of claim 6, including inserting the intramedullary nail into the talus and the tibia through the calcaneus so that the intramedullary nail is free and clear of the calcaneus.

8. The method of claim 7, including making a plantar incision over the guidewire and placing a retractor medial to a sustentaculum to protect a flexor hallucis longus tendon and an adjacent neurovascular bundle prior to the reaming.

9. The method of claim 8, including placing a drill sleeve over the guidewire prior to reaming in order to protect soft tissue.

10. The method of claim 9, including using an outrigger to place a plurality of screws for the fixing of the intramedullary nail to the talus and the tibia.

11. The method of claim 10, including placing a compression screw in the tibia and the talus with the outrigger outside of the intramedullary nail.

12. The method of claim 11, including placing multiple interlocking screws proximally in the nail and the tibia.

13. The method of claim 12, including placing a screw through the intramedullary nail into the talus in a trans malleolar fashion or any other appropriate plane for stability.

14. The method of any of claim 1, including inserting the intramedullary nail into the talus and the tibia through the calcaneus.

15. (canceled)

16. (canceled)

17. (canceled)

18. (canceled)

19. (canceled)

20. A method for fusing a tibiotalar joint of a patient with an intramedullary device while leaving adjacent subtalar joint intact, comprising:
inserting an intramedullary nail through the calcaneus just medial to a main body of a calcaneus at a planar aspect of a sustentaculum tali into a talus and a tibia of the patient without violating a posterior facet of the adjacent talocalcaneal joint; and
fixing the intramedullary nail to the talus and the tibia.

21. (canceled)

22. The method of claim 20, including using a guidewire for placing of the intramedullary nail.

23. The method of claim 22, including starting the guidewire just medial to a main body of a calcaneus at a planar aspect of a sustentaculum tali and inserting the guidewire through the calcaneus into the talus and the tibia.

24. The method of claim 23, including reaming the talus and the tibia over the guidewire by advancing through the sustentaculum tali and into an inferior aspect of the talus without violating the posterior facet of the subtalar joint.

25. The method of claim 24, including making a plantar incision over the guidewire and placing a retractor medial to a sustentaculum to protect a flexor hallucis longus tendon and an adjacent neurovascular bundle prior to the reaming.

26. The method of claim 25, including placing a drill sleeve over the guidewire prior to reaming in order to protect soft tissue.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)
